(19) United States
(12) Patent Application Publication
(10) Pub. No.: US 2007/0151868 A1
(43) Pub. Date: Jul. 5, 2007

(54) IMPLANTABLE ELECTRODE SYSTEM, METHOD AND APPARATUS FOR MEASURING AN ANALYTE CONCENTRATION IN A HUMAN OR ANIMAL BODY

(76) Inventors: Arnulf Staib, Heppenheim (DE); Ralph Gillen, Papenburg (DE)

Correspondence Address: BARNES & THORNBURG LLP, 11 SOUTH MERIDAN STREET, INDIANAPOLIS, IN, 46204, US

(21) Appl. No.: 11/558,933

(22) Filed: Nov. 12, 2006

(30) Foreign Application Priority Data
Nov. 12, 2005 (EP) EP 05024760.0

(51) Int. Cl. G01F 1/64; G01N 33/487

(52) U.S. Cl. 205/792; 204/403.01

(57) ABSTRACT

An electrode system for determining an analyte concentration in a human or animal may comprise first and second electrodes. The first electrode may be configured to produce a first signal from which the analyte concentration can be determined, and may have a first measuring sensitivity that is optimized for a first analyte concentration range. The second electrode may be configured to produce a second signal from which the analyte concentration can be determined, and may have a second measuring sensitivity that is optimized for a second analyte concentration range that is different from the first analyte concentration range. An analytical unit may be configured to determine the analyte concentration based on the first signal if the analyte concentration falls within the first analyte concentration range, and to determine the analyte concentration based on the second signal if the analyte concentration falls within the second analyte concentration range.

### CROSS-REFERENCE TO RELATED APPLICATIONS

This application is a U.S. counterpart application of, and claims priority to, European Application Serial No. EP 05024760.0 filed Nov. 12, 2005, the disclosure of which is incorporated herein by reference.

### FIELD OF THE INVENTION

The invention relates to an implantable electrode system, an apparatus with an electrode system of this type, and a corresponding method for measuring an analyte concentration in a human or animal body. An electrode system of this type is known, for example, from U.S. Pat. No. 6,175,752 B1.

### BACKGROUND

Implantable electrode systems allow measurements of physiologically relevant analytes such as, for example, lactate and glucose, to be made in the body of a patient. Such in vivo measurements are associated with the feasibility of automatic and continuous detection of measuring values.

US 2005/0059871 A1 proposes to measure the analyte concentration of interest using a plurality of electrodes simultaneously and analyze the measuring signals thus obtained by calculation of the mean. As an additional measure, it is recommended to use additional sensors to determine other analyte concentrations or physiological parameters and carry out a plausibility test of the individual results by means of the concentrations of various analytes thus determined.

It is desirable to devise a way for measuring analyte concentrations in a human or animal body at high accuracy.

### SUMMARY

An implantable electrode system for measuring an analyte concentration in a human or animal body may comprise a first and a second measuring electrode for determining measuring signals which each contain information concerning the analyte concentration to be measured, whereby the first measuring electrode has a first measuring sensitivity that is adapted to a first concentration range of the analyte, and the second measuring electrode has a second measuring sensitivity that differs from the first measuring sensitivity and is adapted to a second concentration range of the analyte.

An apparatus for measuring an analyte concentration in a human or animal body may comprise an electrode system of said type, an analytical unit connected to the electrode system for analyzing measuring signals of the first and the second measuring electrode, and a memory, in which at least one first sensitivity parameter characterizing the measuring sensitivity of the first measuring electrode and at least one second sensitivity parameter characterizing the measuring sensitivity of the second measuring electrode are stored, whereby the analytical unit is designed such that analyzing at least one measuring signal of one of the two measuring electrodes allows to determine to which concentration range the analyte concentration belongs, and the measuring signal of the first measuring electrode is analyzed to determine the analyte concentration by means of the first sensitivity parameter if the analyte concentration belongs to the first concentration range, and the measuring signal of the second measuring electrode is analyzed to determine the analyte concentration by means of the second sensitivity parameter if the analyte concentration belongs to the second concentration range.

In an apparatus of this type, the concentration ranges of the measuring electrodes can overlap. It is even possible for the concentration range of one measuring electrode to be a small partial range that is completely included in the significantly larger concentration range of another measuring electrode. It is therefore possible that, for example, the measuring signal of the first measuring electrode is continually analyzed and the measuring signal of the second measuring electrode is analyzed only when the analysis of the first measuring signal shows that the analyte concentration belongs to the concentration range of the second measuring electrode.

A method for measuring an analyte concentration in a human or animal body by means of an implantable electrode system may comprise a first measuring electrode with a first measuring sensitivity and a second measuring electrode with a second measuring sensitivity that differs from the first measuring sensitivity, whereby analyte concentrations belonging to a first concentration range are determined by analyzing a measuring signal of the first measuring electrode and analyte concentrations belonging to a second concentration range are determined by analyzing a measuring signal of the second measuring electrode.

Varying analyte concentrations in a human or animal body generally cannot be determined with a single measuring electrode without limitations in the measuring accuracy. This is because, typically, the larger the measuring range of a measuring electrode is selected, the lower is the measuring sensitivity at low concentrations. Instead of designing a measuring electrode by striving for an optimal compromise between a largest-possible measuring range and a highest-possible measuring sensitivity, a large measuring range may be attained by the use of a plurality of measuring electrodes that cover different measuring ranges and accordingly have different measuring sensitivities. A larger measuring range can be implemented without limiting the measuring accuracy by the measuring sensitivity of the first measuring electrode being optimized for a first concentration range and the measuring sensitivity of the second measuring electrode being optimized for a second concentration range.

Therefore, the analyte concentration can always be measured with a measuring electrode with a favorable measuring sensitivity for the corresponding concentration range regardless of whether the analyte concentration in the vicinity of the measuring electrodes is relatively high or low with respect to a nominal or average physiological value.

It is feasible to use a plurality of first measuring electrodes for an upper concentration range and a plurality of second measuring electrodes for a lower concentration range in order to reduce the susceptibility of the system to interference or further improve the measuring accuracy by means of a statistical analysis.

If a plurality of first or a plurality of second measuring electrodes is used in an electrode system, a statistical analysis for improvement of the measuring accuracy requires the individual measuring electrodes to be triggered separately. In an electrode system according to the invention, though, a plurality of identical measuring electrodes can also be arranged in parallel circuitry with common triggering such that the current signal can be increased and the signal-to-noise ratio can be improved even in the absence of a statistical analysis.

If no use is made of a plurality of identical measuring electrodes, it is desirable to select the area of the measuring electrode with the lower measuring sensitivity to be larger, preferably at least 50% larger, than the area of the measuring electrode with the (next) higher measuring sensitivity. This allows an increased current signal to be obtained and the signal-to-noise ratio thus to be improved even at a lower measuring sensitivity.

### BRIEF DESCRIPTION OF THE DRAWINGS

The invention shall be illustrated in more detail in the following based on exemplary embodiments that are shown in the figures. The particularities shown therein can be used individually or in combination to create further developments. Equal or equivalent components are identified by consistent reference numbering. In the figures:

FIG. 1 shows a flow diagram for determining an analyte concentration using an electrode system according to the invention;

FIG. 2 shows an exemplary embodiment of an electrode system according to the invention;

FIG. 3 shows another exemplary embodiment of an electrode system according to the invention;

FIG. 4 shows a cross-sectional view of the electrode system shown in FIG. 3;

FIG. 5 shows an example of a characteristic curve of a first measuring electrode of an electrode system according to the invention;

FIG. 6 shows an example of a characteristic curve of a second measuring electrode of an electrode system according to the invention; and

FIG. 7 shows a schematic view of an apparatus according to the invention for measuring an analyte concentration using an electrode system according to the invention.

### DESCRIPTION OF THE ILLUSTRATIVE EMBODIMENTS

Glucose is an example of an analyte whose concentration in blood and other body fluids of a patient can be subject to strong variation, such as between 40 mg/dl and 450 mg/dl, during the course of a day. The determination of analyte concentrations shall be illustrated in the following using measurements of the glucose concentrations in blood or interstitial fluid as an example without limiting the scope of the invention.

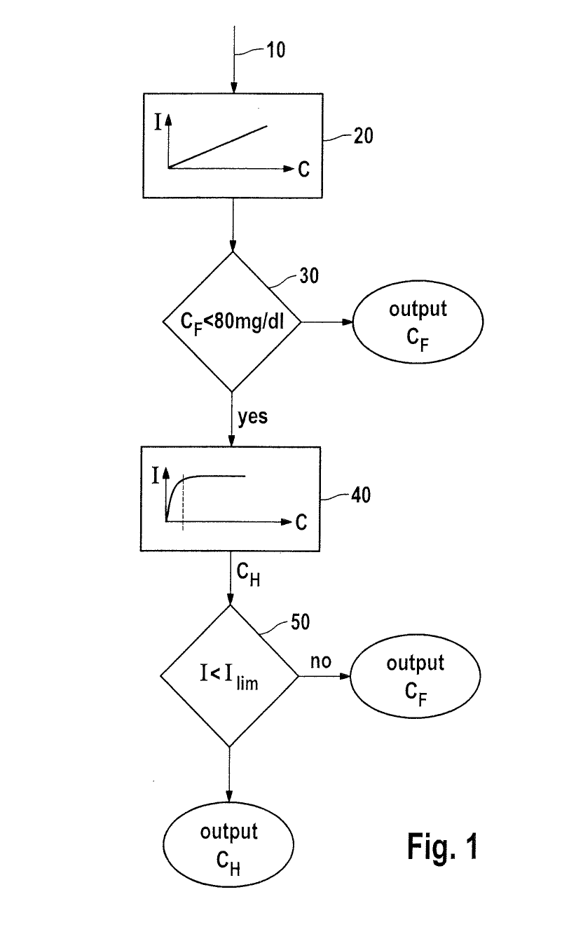

FIG. 1 shows a flow diagram for determining the glucose concentration using an implantable electrode system that comprises two galvanically separated measuring electrodes with different sensitivities. A first measuring electrode has a first measuring sensitivity that is optimized for a first concentration range of the analyte ranging from, for example, 80 mg/dl to 500 mg/dl. A second measuring electrode has a second measuring sensitivity that is optimized for a second concentration range of the analyte ranging from, for example, 20 mg/dl to 80 mg/dl. Accordingly, the first concentration range differs from the second concentration range. It is desirable for the upper thresholds of the concentration ranges to differ at least by a factor of 2.

The measuring electrodes each provide a measuring signal in the form of, for example, an electrical current whose amplitude depends on the analyte concentration in the vicinity of the measuring electrodes. For each measuring electrode, a characteristic curve, that may be characterized by a sensitivity parameter, describes the relationship between the current I, as the measuring signal, and the corresponding analyte concentration C.

In order to determine the analyte concentration, the measuring signal of the first measuring electrode is analyzed by means of a characteristic curve 20 and a value C_{F} reflecting the analyte concentration is determined in a first working step 10. In a subsequent step 30, a check is made whether or not the value C_{F} determined by means of the measuring signal of the first measuring electrode belongs to the concentration range for which the measuring sensitivity of the first measuring electrode is optimized. In the exemplary embodiment shown, the first measuring electrode is optimized for analyte concentrations in excess of 80 mg/dl. If the value C_{F} is thus determined to belong to the first concentration range, i.e. to exceed 80 mg/dl, the value C_{F} is output as the result.

If the concentration value C_{F} determined by means of the first measuring electrode is less than 80 mg/dl, the measuring signal of the second measuring electrode is analyzed by means of the characteristic curve 40 of the second measuring electrode.

Illustratively, the characteristic curve 40 of the second measuring electrode has a steepest-possible slope at low concentration values such that a signal-to-noise ratio that is as high as possible results for low concentrations. Since the maximal current that is possible as measuring signal is limited, saturation effects cause a constant or virtually constant maximal current I_{Iim} at higher concentrations. In contrast, the characteristic curve 20 of the first measuring electrode has a close-to-linear shape such that even high analyte concentrations can be determined reliably without interference from saturation effects. Whereas, an analysis of low measuring currents I corresponding to low concentrations by means of the first characteristic curve 20 is made difficult by an unfavorable signal-to-noise ratio, measuring currents I exceeding a threshold value I_{Iim} cannot be analyzed reasonably by means of the second characteristic curve 40 due to saturation effects.

For this reason, a check is made in a working step 50 whether or not the measuring current I of the second measuring electrode is lower than a given threshold value I_{Iim}. If this is the case, then a concentration value C_{H} obtained by analyzing the measuring signal of the second measuring electrode is more reliable than the concentration value C_{F} determined by means of the first measuring electrode such that the concentration value C_{H} is output as the result. However, if the measuring current I of the second measuring electrode is higher than or equal to the threshold value I_{Iim}, then the concentration value C_{F} determined by means of the first measuring electrode is more reliable than the concentration value C_{H} determined by means of the second measuring electrode such that the value C_{F} is output as concentration value.

In principle, the working step 50 is dispensable if the threshold value checked in working step 30, being 80 mg/dl in the example shown, is selected suitably. However, after implantation of an electrode system there may occur changes in the measuring sensitivity of the individual measuring electrodes such that the saturation range of the second measuring electrode starts at lower concentrations. This can be recognized in the working step 50 and the concentration range in which analyte concentrations are determined using the first measuring electrode can be extended somewhat to include lower analyte concentrations, if applicable. It is also feasible to select the concentration range for which the first measuring electrode is optimized and the concentration range for which the second measuring electrode is optimized to be overlapping and to determine the analyte concentration in an area of overlap by means of a statistical analysis from an analytical result of the first measuring electrode and an analytical result of the second measuring electrode. For example, the area of overlap can be selected to range from 70 mg/dl to 100 mg/dl. The statistical weights for weighting the results of the first and the second measuring electrode in the area of overlap can be selected taking into consideration the signal-to-noise ratio of the respective measuring signal.

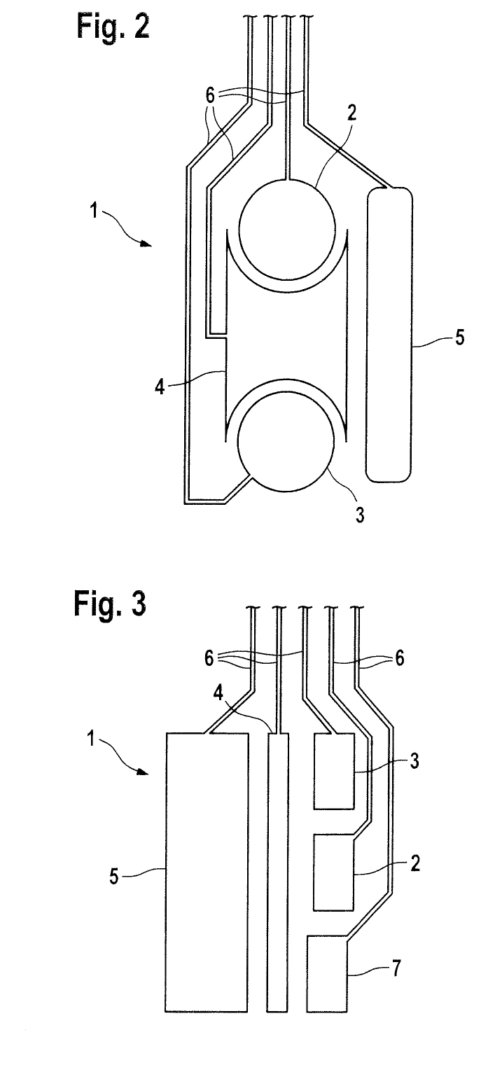

FIG. 2 shows an exemplary embodiment of an electrode system 1 that can be used to carry out the method described. The electrode system 1 comprises a first measuring electrode 2 and a second measuring electrode 3 which each have different measuring sensitivities. A common counter-electrode 4 is associated with the two measuring electrodes 2, 3 that is at earth potential when in operation such that a first measuring current I1 flows between the first measuring electrode 2 and the counter-electrode 4 and a second measuring current I2 flows between the second measuring electrode 3 and the counter-electrode 4. The electrode system 1 further comprises a reference electrode 5 that provides a reference potential for the measuring electrodes 2, 3. The reference potential is preferably defined by silver/silver chloride redox reaction, although other redox reactions may also be used for the reference electrode.

In principle, it is feasible to dispense with a separate reference electrode 5 and use the counter-electrode 4 as reference electrode also. However, since the measuring currents I1 and I2 flow through the counter-electrode 4, this would cause the redox reaction defining the reference potential (for example silver/silver chloride) to eventually cease effectively limiting the serviceable life of the electrode system. Using a separate reference electrode 5, the reference potential can be provided in the absence of any flow of current such that the serviceable life of the electrode system is not limited in this respect.

FIG. 3 shows another exemplary embodiment of an implantable electrode system 1 that differs from the exemplary embodiment shown in FIG. 2, aside from the shape and arrangement of the individual electrodes, in that a total of three measuring electrodes 2, 3, 7 are provided. Similar to the electrode system 1 shown in FIG. 2, the second measuring electrode 3 serves for measuring glucose concentrations of less than, for example, 80 mg/dl. Whereas the first measuring electrode 2 is used for the entire concentration range above 80 mg/dl in the exemplary embodiment shown in FIG. 2, a third measuring electrode for concentrations in excess of, for example, 300 mg/dl is provided in the electrode system 1 shown in FIG. 3.

In particular with regard to the use of more than two measuring electrodes it is favorable to select the measuring sensitivities of the individual measuring electrodes such that they result in overlapping measuring ranges. Plausibility tests in the area of overlap become feasible if, for example, the first concentration range for which the measuring sensitivity of the first measuring electrode 2 is optimized overlaps with the second concentration range for which the measuring sensitivity of the second measuring electrode 3 is optimized. Moreover, analyte concentrations in the area or areas of overlap can be determined by a statistical analysis from analytical results of various measuring electrodes.

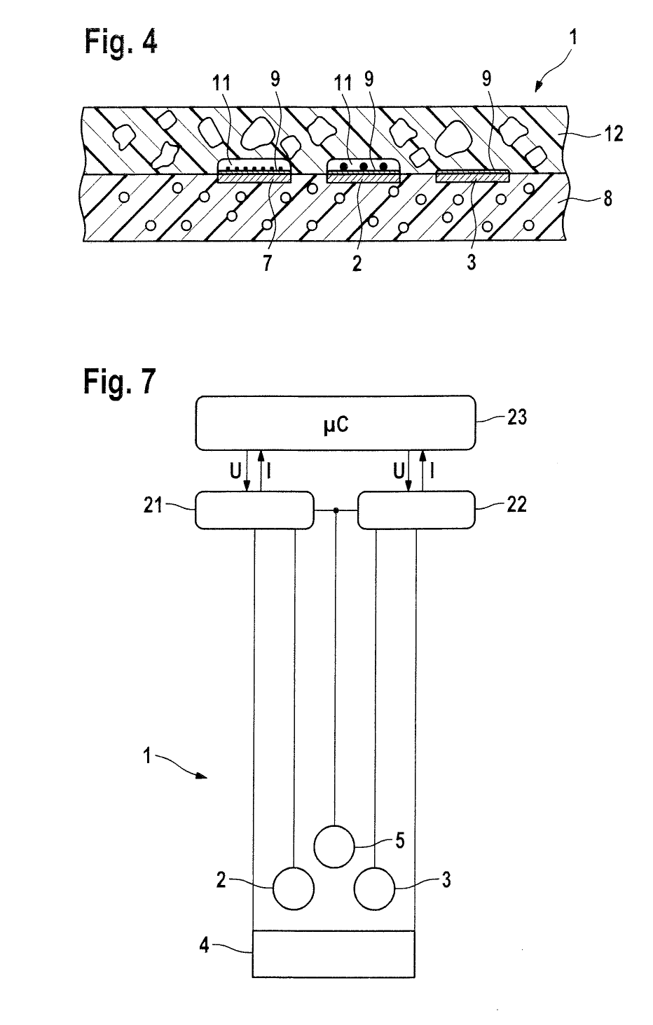

FIG. 4 shows a cross-section of the electrode system 1 shown in FIG. 3, whereby the sectional line extends through the measuring electrodes 2, 3, and 7. The electrodes 2, 3, 4, 5, and 7 are arranged on a common carrier 8 illustratively made of plastic material, for example of polyimide, and contacted by printed conductors 6 via which the electrode system 1 can be connected to an analytical unit (not shown). In operation, the analytical unit sets the potential of the measuring electrodes 2, 3, 7 to a predefined value of, for example, 350 mV with respect to the reference electrode 5. Although the measuring electrodes drift with respect to the earth potential of the counter electrode 4, for example by deposition of proteins after implantation, this allows defined conditions for precise analysis of the measuring currents to be created, since the measuring electrodes 2, 3, 7 and the reference electrode 5 can be presumed to be subject to the same drifting effects.

Illustratively, the measuring electrodes 2, 3, 7 each have an area of 0.1 mm² to 0.5 mm² and, like the printed conductors 6, are made of gold. The measuring electrodes 2, 3, 7 each are coated with an enzyme layer 9 containing an enzyme that generates, by catalytic conversion of the analyte, charge carriers that are detected to generate the measuring signal. The charge carriers can be generated either directly or by conversion of an intermediate product initially generated by the enzyme. An example of an intermediate product of this type is hydrogen peroxide. In the exemplary embodiment shown, the enzyme layers 9 are provided in the form of carbon layers immobilizing the enzyme, e.g., glucose oxidase. The enzyme layers have a thickness of, for example, 3 μm to 10 μm, or illustratively 5 μm.

The different measuring sensitivities of the measuring electrodes 2, 3, 7 are illustratively generated by at least one of the measuring electrodes 2, 3, 7 comprising a covering layer 11 that produces a diffusion resistance for the analyte, whereby a difference between the measuring sensitivities of the measuring electrodes 2, 3, and 7 is implemented by adapting the diffusion resistance. Since the diffusion resistance of a covering layer 11 is larger the thicker the respective covering layer 11 is, different diffusion resistances can be implemented most easily by covering layers 11 differing in thickness.

For example, for this purpose, a covering membrane of 30 μm in thickness made from a poorly swelling polymer, for example polyurethane, can be applied to the first measuring electrode 2, and a covering membrane made from the same material that is only 10 μm in thickness can be applied to the second measuring electrode 3. Due to the diffusion resistance of the covering layers of the measuring electrodes 2, 3 being different, different numbers of glucose molecules reach the enzyme layers of the measuring electrodes 2,3 per unit time. The glucose oxidase enzyme stored in the enzyme layer degrades the glucose molecules such that charge carriers are released that are detected at the measuring electrodes 2, 3 and thus form the measuring currents I1 and I2. In the simplest case, different diffusion resistances can be implemented by means of differences in the thickness of the covering layers of the measuring electrodes 2, 3.

Another option is to provide for differences in the microstructure of the covering layers, for example their porosity, or to manufacture the covering layers from different materials. For example, silicone, being relatively impermeable for glucose molecules, can be used for the covering layer 11 of the measuring electrode 7 for high concentrations and polyurethane, being relatively permeable for glucose molecules, can be used for the covering layer 11 of the measuring electrode 3 for medium concentrations, and a covering layer can be dispensed with in the case of measuring electrode 2.

Poorly swelling polymers, such as polyurethane for example, are particularly favorable for the covering layer 11. The covering membrane is illustratively less than 50 μm, and in a particular embodiment 10 μm to 30 μm in thickness.

Due to the diffusion resistance of the covering layers 11 of the measuring electrodes 2, 3 being different, different numbers of glucose molecules reach the enzyme layers of the measuring electrodes 2,3 per unit time. The glucose oxidase enzyme stored in the enzyme layer degrades the glucose molecules such that charge carriers are released that are detected at the measuring electrodes 2, 3 and thus form different measuring currents.

Another option for implementing different measuring sensitivities for the first and second measuring electrode 2, 3 is to use different enzyme layers 9. For example, in order to increase the measuring sensitivity of the first measuring electrode 2, the amount of enzyme in the enzyme layer 9 can be selected to be twice as high as for the enzyme layer 9 of the second measuring electrode 3.

The measuring electrodes 2, 3, 7 are covered by a dialysis membrane 12 which preferably extends over the entire surface of the electrode system 1. In this context, a dialysis membrane 12 shall be defined to mean a membrane that is impermeable for molecules larger than a certain maximal size. Illustratively, the dialysis membrane 12 is pre-manufactured in a separate manufacturing process and applied as a complete finished structure during the manufacture of the electrode system 1. The maximal size of the dialysis membrane is selected for the electrode system 1 shown such that analyte molecules can permeate through the dialysis membrane 12 while larger molecules are retained. In the exemplary embodiment shown, the dialysis membrane 12 is provided in the form of a porous layer made from a suitable plastic material, in particular polyarylethersulphone. The use of a dialysis membrane 12 allows the effective surface of the measuring electrodes 2, 3, 7 to be enlarged and thus the signal-to-noise ratio to be improved. However, in principle, it is feasible to dispense with a dialysis membrane as long as all measuring electrodes are provided with a covering layer 11.

For the electrode system 1 described above to function properly, it is desirable for all measuring electrodes 2, 3, 7 to be exposed to the same analyte concentration. Therefore, the individual measuring electrodes 2, 3, 7 illustratively are separated from each other by less than 1.5 mm, e.g., less than 1 mm, and in one particular embodiment less than 700 μm. If a plurality of measuring electrodes is used, it is therefore desirable to ensure that the distance between the electrodes, even the ones farthest from each other, is not too large. Homogeneous analyte concentrations in the area of the electrode system 1 can be effected, for example, by a sufficiently thick dialysis membrane. The thickness of the dialysis membrane is therefore illustratively 50 μm to 500 μm, and in one particular embodiment 100 μm to 300 μm.

Another option for homogenizing the analyte concentrations in the area of the electrode system 1 is to arrange the dialysis membrane not directly on the measuring electrodes 2, 3, 7, but rather to arrange the measuring electrodes 2, 3, 7 in a small chamber that is sealed off by the dialysis membrane. A chamber of this type can be implemented, for example, by arranging the measuring electrodes 2, 3, 7 in a suitable recess of the carrier 8 and covering the recess with the dialysis membrane 12. The height of a chamber of this type, i.e. the distance from the surface of the measuring electrodes 2, 3, 7 (or their covering layer 11) to the bottom side of the dialysis membrane 12 illustratively is less than 400 μm, and in one particular embodiment less than 300 μm.

Like a thick dialysis membrane, a chamber of this type serves as a reservoir for the analyte such that a transient lateral blockade of the dialysis membrane 12 can be evened out.

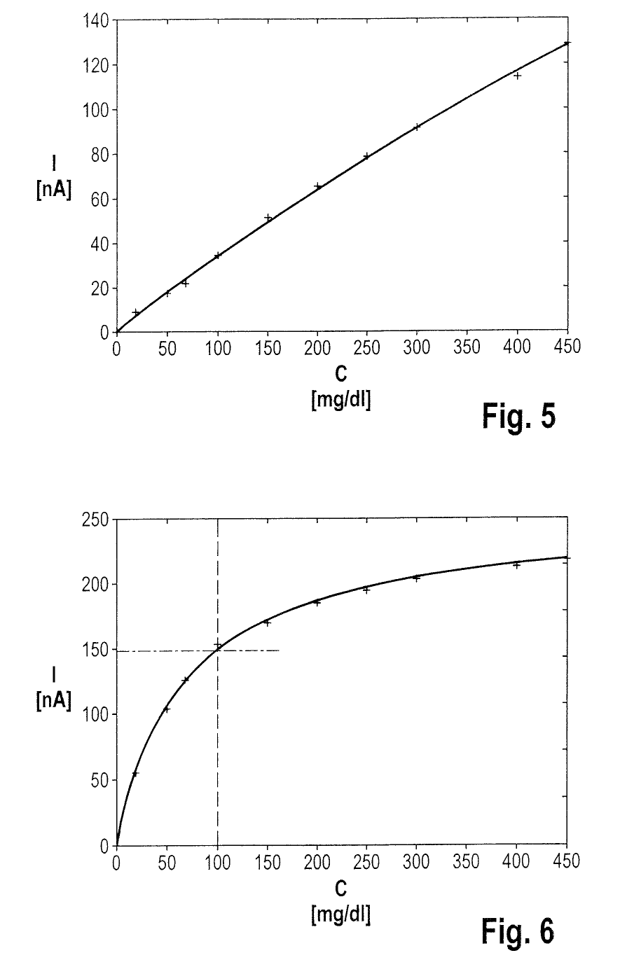

FIG. 5 shows in more detail the characteristic curve 20 previously illustrated in the context of FIG. 1, whereby said characteristic curve represents the functional dependence of the current I representing the measuring signal of the first measuring electrode 2 to the analyte concentration C. In practical application, the measuring accuracy in the determination of high analyte concentrations is limited by saturation effects. In order to optimize the measuring sensitivity of the first measuring electrode for high analyte concentrations, it is therefore favorable to strive for the shape of the characteristic curve to be as close to linear as possible over the entire physiologically-relevant concentration range.

Glucose concentrations in excess of, for example, 450 mg/dl virtually never occur in the human body such that the shape of the characteristic line shown in FIG. 5 above 450 mg/dl is irrelevant. If a linear relationship exists between the current I and the concentration C, the sensitivity of the measuring electrode can be characterized by a single sensitivity parameter with the parameter being the derivative of the characteristic curve with respect to concentration, i.e. the slope. In practical application, a perfectly linear relationship usually cannot be implemented such that additional sensitivity parameters are needed in order to describe the shape of the characteristic line.

In order to render the influence of saturation effects as negligible as possible, the sensitivity of the first measuring electrode 2 at 450 mg/dl should illustratively be at least 80% of the sensitivity at 100 mg/dl, such as is the case with the characteristic curve shown in FIG. 5. Moreover, the sensitivity at 100 mg/dl should illustratively be at least 0.1 nA/mg/dl such that concentrations in excess of 100 mg/dl can be detected at a sufficiently high signal-to-noise ratio. With regard to concentrations of less than approximately 100 mg/dl, the characteristic line shown in FIG. 5 leads to an increasingly unfavorable signal-to-noise ratio such that the measuring accuracy for low concentrations is insufficient.

For this reason, a second measuring electrode with significantly higher sensitivity in this low concentration range is used for the measuring of analyte concentrations of less than, for example, 80 mg/dl. An example of the characteristic line of a suitable measuring electrode is shown in FIG. 6. In order for the measuring sensitivity to be as high as possible at low concentrations, the characteristic curve must be as steep as possible in the respective concentration range. Since the measuring currents attainable with implanted electrodes are limited, high measuring sensitivity at low concentrations is associated with saturation at higher concentrations. This is evident in FIG. 6 from a marked flattening of the characteristic curve at concentrations in excess of 200 mg/dl. This results in a very unfavorable signal-to-noise ratio at high concentrations as opposed to a very favorable signal-to-noise ratio at low concentrations.

The measuring sensitivities of the first measuring electrode and the second measuring electrode at a reference concentration, for example a glucose concentration of 100 mg/dl, illustratively differ by at least a factor of 2, and in one particular embodiment by at least a factor of 3. Illustratively, a concentration belonging to the first or second concentration range is selected as reference concentration, for example the arithmetic mean of the upper threshold and lower threshold of one of the concentration ranges. In this context, the measuring sensitivity is the derivative of the intensity of the measuring signal with respect to concentration, i.e. the slope of the characteristic curve at the corresponding concentration.

In order for critical glucose concentrations of 50 mg/dl to be reliably detected using the second measuring electrode, the sensitivity of the second measuring electrode between 10 mg/dl and 100 mg/dl should be at least 1 nA/mg/dl. This results in currents of at least 50 nA at a critical glucose concentration of 50 mg/dl such that a signal-to-noise ratio of 5 or better can be attained.

FIG. 7 shows a schematic view of an apparatus for measuring an analyte concentration using an electrode system 1. In this context, the electrode system 1 described above is connected to an analytical unit 23 that is provided as a microprocessor and comprises a memory, in which at least one first sensitivity parameter characterizing the measuring sensitivity of the first measuring electrode 2 and at least one second sensitivity parameter characterizing the measuring sensitivity of the second measuring electrode 3 are stored. By analyzing at least one measuring signal of one of the measuring electrodes 2, 3, the analytical unit 23 determines in which concentration range the analyte concentration in the vicinity of the electrode system 1 is. If the analyte concentration belongs to the concentration range for which the measuring sensitivity of the first measuring electrode 2 is optimized, the analyte concentration is determined by analyzing the measuring signal of the first measuring electrode 2. If the analyte concentration belongs to the concentration range for which the measuring sensitivity of the second measuring electrode 3 is optimized, the analyte concentration is determined by analyzing the measuring signal of the second measuring electrode 3.

If the concentration ranges of the individual measuring electrodes 2, 3 overlap, analyte concentrations belonging to an area of overlap can also be determined by analyzing the measuring signals of two measuring electrodes 2, 3.

The first measuring electrode 2 is connected to a first potentiostat 21 and the second measuring electrode 3 is connected to a second potentiostat 22. The potentiostats 21, 22 are each triggered by the microprocessor providing the control and analytical unit 23. A potentiostat is an electronic control circuit that is used to set to a desired value the potential that is applied to the respective measuring electrode 2, 3 with regard to a reference electrode 5. The current to be measured flows between the measuring electrodes 2, 3 on which the enzymatic conversion of the analyte and generation of charge carriers proceed upon adequate setting of the potential, and the counter-electrode 4. The reference point is represented by the reference electrode 5 whose potential is defined in the electrochemical series. Illustratively, no current flows through the reference electrode 5 in this process.

The maximal measuring current in an implanted electrode system is very limited since the charge carriers required for the measuring current are generated by enzymatic degradation of the analyte. Therefore, due to the influence of the measuring current and the enzyme employed, the analyte concentration in the vicinity of the electrode system 1 that is relevant for the measurement is at risk of being depleted, in particular if the body tissue surrounding the measuring electrodes 2, 3 is perfused relatively poorly by body fluid or if the transport of analyte molecules in the vicinity of the measuring electrodes 2, 3 is inhibited for other reasons, for example by a blockade. The quantity of analyte typically consumed per minute by a measuring electrode in continuous measuring operation is in the picomol range, i.e. relatively small. Nevertheless, the depletion of the analyte concentration in the area of the measuring electrodes 2, 3 can lead to an incorrect measurement. In the most unfavorable case, this effect can lead to a physiologically plausible decrease in signal intensity, i.e. feign a reduction of analyte concentration throughout the body of the patient.

The apparatus shown in FIG. 7 can be used to check whether a decrease in glucose concentration that is observed over a certain period of time using the electrode system 1 has a natural cause and therefore occurs throughout the body of the patient, or occurs only locally in the vicinity of the electrode system. A non-physiological contribution to a decrease in signal intensity that is caused by local depletion of the glucose concentration in the vicinity of the measuring electrodes 2, 3 is recognized by applying a measuring voltage to the measuring electrodes 2, 3 in an alternating fashion. If the measuring electrodes 2, 3 are turned on and off separately from each other over a period of several minutes as is relevant for the detection of depletion, the profile of the signal can be tested for each measuring electrode separately. It is a sign of depletion if, in the process, a more rapid decrease in signal intensity is detected for the measuring electrode with the higher measuring sensitivity as compared to the measuring electrode with the lower measuring sensitivity.

A simple algorithm, for example a linear compensation calculation or a non-linear curve analysis, allows characteristic signal decrease times to be determined for each of the measuring currents of the measuring electrodes 2, 3, whereby a numerical adjustment can be made if an equilibrium is established. If, in the process, the analytical unit 23 determines different characteristic signal decrease times for the measuring electrodes 2, 3 within a time period that is relevant for depletion, an alarm signal can be used to alert to the unreliability of the calculated glucose concentration.

As long as the depletion thus determined does not exceed a critical threshold value, there is the option to numerically compensate for the measuring current-effected depletion of the analyte concentration in the analysis of the measuring currents for determining the true analyte concentration in the body of the patient. For example, the consumption kinetics of the charge carriers with respect to the analyte can be described by a simple model. The signal decrease times determined by the analytical unit 23 based on the current measurements can be compared to theoretical values of the model, and thus provide the basis for a numerical compensation of the local analyte concentration determined from the measuring values.

Accordingly, in the exemplary embodiment shown, the analytical unit 23 carries out a processing procedure during the analysis of the measuring signals. In this procedure, correction data pertaining to a measuring current-effected local decrease of the analyte concentration is taken into consideration, whereby, in order to obtain the correction data, the measuring voltages applied to the measuring electrodes 2, 3 are turned on and off separately and the time profile of the measuring currents is analyzed, in particular by determining characteristic signal decrease times of the measuring currents. In the simplest case, this correction data can be the characteristic signal decrease times.

In the exemplary embodiment shown in FIG. 7, the microprocessor 23 provides both the analytical unit for analysis of the measuring signals and the control unit that allows the respective measuring voltages applied to the measuring electrodes 2, 3 to be turned on and off separately. The analytical unit 23 is designed such that a local decrease of the analyte concentration, effected by a measuring current for at least one of the measuring electrodes 2, 3 after the measuring voltage is turned on, is recognized and taken into consideration in the analysis for determining the analyte concentration in the human or animal body. This can be taken into consideration by indicating by means of a signal that the analyte concentration cannot be determined at the present time. Another option is to quantitatively detect the local decrease of the analyte concentration and compensate for it numerically in the calculation of the analyte concentration in the body of the patient.

The analytical and control unit 23 can, for example, also serve for controlling an artificial pancreas or can be connected to a display facility that is used to display analyte concentrations thus determined and/or to alert a patient to particularly high or low analyte concentrations necessitating counter-measures, by means of a suitable signal, for example an acoustic signal. In this context, it is particularly favorable for the data transmission to the display facility to proceed in a wireless fashion, for example by means of an infrared interface or by means of ultrasound.

1-13. (canceled)

14. An electrode system for determining an analyte concentration in a human or animal, comprising:
a first electrode configured to produce a first signal from which the analyte concentration can be determined, the first electrode having a first measuring sensitivity that is optimized for a first analyte concentration range, and
a second electrode configured to produce a second signal from which the analyte concentration can be determined, the second electrode having a second measuring sensitivity that is optimized for a second analyte concentration range that is different from the first analyte concentration range.

15. The electrode system of claim 14 wherein the first and second electrodes are configured such that the first and second concentration ranges do not overlap.

16. The electrode system of claim 14 wherein the first and second electrodes are configured such that the first and second concentration ranges overlap.

17. The electrode system of claim 14 wherein the first and second electrodes are configured such that the first and second measuring sensitivities differ at a reference concentration by at least a factor of 2.

18. The electrode system of claim 14 wherein the first and second electrodes each comprise an enzyme layer having an enzyme that generates, by catalytic conversion of the analyte, charge carriers that are captured for generating the first and second signals.

19. The electrode system of claim 18 wherein the enzyme layer comprising the first electrode is a different enzyme layer than the enzyme layer comprising the second electrode.

20. The electrode system of claim 18 wherein the enzyme layer comprising one of the first and second electrodes is provided in a different quantity than the enzyme layer comprising the other of the first and second electrodes.

21. The electrode system of claim 14 wherein one of the first and second electrodes comprises a covering layer that produces a diffusion resistance for the analyte,
and wherein a difference in the first and second measuring sensitivities results from the covering layer comprising the one of the first and second electrodes.

22. The electrode system of claim 14 further comprising a counter electrode that is common to the first and second electrodes.

23. The electrode system of claim 14 further comprising a third electrode configured to produce a third signal from which the analyte concentration can be determined, the third electrode having a third measuring sensitivity that is optimized for a third analyte concentration range that is different from the first and second analyte concentration ranges.

24. The electrode system of claim 14 further comprising an analytical unit configured to analyze the first and second signals to determine whether the analyte concentration falls within the first analyte concentration range or the second analyte concentration range.

25. The electrode system of claim 24 wherein the analytical unit is configured to determine the analyte concentration based on the first signal if the analyte concentration falls within the first analyte concentration range, and to determine the analyte concentration based on the second signal if the analyte concentration falls within the second analyte concentration range.

26. The electrode system of claim 24 wherein the first and second analyte concentrations overlap in an area of overlap,
and wherein the analytical unit is configured to determine the analyte concentration in the area of overlap based on a statistical analysis of a first analyte concentration determined from the first signal and a second analyte concentration determined from the second signal.

27. The electrode system of claim 14 further comprising:
an analytical unit configured to receive the first and second signals, and
a memory in which at least one first sensitivity parameter defining the first measuring sensitivity and at least one second sensitivity parameter defining the second measuring sensitivity are stored,
wherein the analytical unit is configured to determine from at least one of the first and second signals whether the analyte concentration belongs in the first or the second analyte concentration range,
and wherein the analytical unit is configured to determine the analyte concentration based on the first signal and the at least one first sensitivity parameter if the analyte concentration belongs in the first analyte concentration range, and based on the second signal and the at least one second sensitivity parameter if the analyte concentration belongs in the second analyte concentration range.

28. The electrode system of claim 27 wherein the first and second analyte concentration ranges overlap in an area of overlap,
and wherein the analytical unit is configured to determine the analyte concentration in the area of overlap based on a statistical analysis of a first analyte concentration determined from the first signal and the at least one first sensitivity parameter and a second analyte concentration determined from the second signal and the at least one second sensitivity parameter.

29. The electrode system of claim 27 further comprising:
a first potentiostat electrically connected between the first electrode and the analytical unit, and
a second potentiostat electrically connected between the second electrode and the analytical unit.

30. The electrode system of claim 29 wherein the analytical unit comprises a processor configured to separately control each of the first and second potentiostats to selectively apply and remove measuring voltages to each of the first and second electrodes,
and wherein the analytical unit is configured to compensate numerically for a local decrease in the analyte concentration resulting from a measuring current for at least one of the first and second electrodes after applying the measuring voltage to the at least one of the first and second electrodes.

31. A method for determining analyte concentration in a human or animal, the method comprising:
receiving a first measurement signal from a first implanted electrode that is optimized to produce first measurement signals over a first analyte concentration range,
receiving a second measurement signal from a second implanted electrode that is optimized to produce second measurement signals over a second analyte concentration range that is different from the first analyte concentration range, and
processing at least one of the first and second measurement signals to determine the analyte concentration.

32. The method of claim 31 wherein processing at least one of the first and second measurement signals comprises:
processing at least one of the first and second measurement signals to determine whether the analyte concentration falls within the first or second analyte concentration range,
determining the analyte concentration based on the first measurement signal if the analyte concentration falls within the first analyte concentration range, and
determining the analyte concentration based on the second measurement signal if the analyte concentration falls within the second analyte concentration range.

33. The method of claim 31 wherein processing at least one of the first and second measurement signals comprises,
processing at least one of the first and second measurement signals to determine whether the analyte concentration falls within an overlap area defined between the first and second analyte concentration ranges,
if the analyte concentration falls within the overlap area, determining a first analyte concentration based on the first measurement signal, determining a second analyte concentration based on the second measurement signal and determining the analyte concentration based on a statistical analysis of the first and second analyte concentrations.

34. The method of claim 31 wherein the first and second analyte concentrations ranges overlap.

35. The method of claim 31 wherein the first and second analyte concentration ranges do not overlap.

36. The method of claim 31 wherein the first implanted electrode has a first measuring sensitivity that is optimized to produce the first measurement signals over the first analyte concentration range,
and wherein the second implanted electrode has a second measuring sensitivity that is optimized to produce the second measurement signals over the second analyte concentration range,
and wherein the first and second sensitivities differ by at least a factor of 2.

37. The method of claim 31 further comprising receiving a third measurement signal from a third implanted electrode that is optimized to produce third measurement signals over a third range of analyte concentrations that is different from the first and second analyte concentration ranges, and
and wherein processing at least one of the first and second measurement signals comprises processing at least one of the first, second and third measurement signals to determine the analyte concentration.
